(19) United States
(12) Patent Application Publication
(10) Pub. No.: US 2024/0041724 A1
(43) Pub. Date: Feb. 8, 2024

(54) REDUCTION OF STICKINESS OF COSMETIC COMPOSITIONS COMPRISING BISOCTRIZOLE

(71) Applicant: DSM IP ASSETS B.V., HEERLEN (NL)

(72) Inventors: Cyrille DESHAYES, Kaiseraugst (CH); Christine MENDROK-EDINGER, Kaiseraugst (CH)

(21) Appl. No.: 18/256,161

(22) PCT Filed: Dec. 8, 2021
(86) PCT No.: PCT/EP2021/084825

§ 371 Date: Jun. 6, 2023

(30) Foreign Application Priority Data
Dec. 9, 2020 (EP) 20212803.9

(51) Int. Cl. A61K 8/31; A61K 8/49; A61K 8/60; A61K 8/37; A61K 8/04; A61Q 17/04

(52) U.S. Cl. CPC A61K 8/31; A61K 8/496; A61K 8/602; A61K 8/37; A61K 8/042; A61Q 17/04; A61K 2800/5922

(57) ABSTRACT

The present invention relates to cosmetic composition comprising the UV-filter Bisoctrizole, a C8-C16 alkyl poly-glucoside, a mixture of branched and linear saturated C15-C19, wherein said mixture comprised primarily branched saturated C15-C19, and an specific dialkyl ether or (di)ester. This cosmetic composition shows a reduced stickiness, particular a reduced adherence of sand onto the skin to which a composition comprising the UV-Filter Bisoctrizole and C8-C16 alkyl poly-glucoside has been applied. This reduction of stickiness is essential to formulate cosmetic compositions having a high sun protection factor.

### TECHNICAL FIELD

The present invention relates to the field of cosmetic compositions which protect from UV light, particularly to cosmetic compositions comprising the UV-filter Bisoctrizole.

### BACKGROUND OF THE INVENTION

The trend away from elegant pallor towards “healthy, sporty brown skin” has been unbroken for years. In order to achieve this, people expose their skin to the sun's rays, as this causes pigment formation in the sense of melanin formation. However, the ultraviolet radiation of sunlight also has a damaging effect on the skin. In addition to acute damage (sunburn), long-term damage such as an increased risk of skin cancer occurs if the skin is excessively exposed to light from the UV(B) range (wavelength: 280-315 nm). Excessive exposure to UV(B) and UV(A) radiation (wavelength: 315-400 nm) also weakens the elastin and collagen fibres of the connective tissue. This leads to numerous phototoxic and photoallergic reactions and results in premature skin ageing.

To protect the skin, a series of light protection filter substances have therefore been developed which can be used in cosmetic preparations.

Bisoctrizole is a very important UV-filter which is broadly used and has excellent UV absorption properties. The solubility of Bisoctrizole, however, is very low in the most solvents and cosmetic oils. Therefore, Bisoctrizole is typically used as micronized powder as an aqueous suspension which is stabilized by alkyl glucoside. However, this leads to the fact that the product is rather high viscous. As a result of this, the cosmetic composition comprising Bisoctrizole have a certain stickiness when applied to the skin. This stickiness increases the higher the amount of Bisoctrizole is to achieve higher sun protection. This stickiness is particularly pronounced when the cosmetic composition is used on the beach, as it leads to sand adhering to creamed areas of skin, an adverse effect consumers suffer from at the beach. To prevent this, consumers tend to use less sunscreen but at the same time they are less protected. The adverse effects of UV radiation on skin are well known. It is therefore important for the industry to provide solutions for such kind of issues to guarantee, that consumers apply sufficient sunscreen for appropriate protection.

Emollients, particularly ether and ester based emollients are used frequently to obtain the desired application properties of cosmetic compositions.

### SUMMARY OF THE INVENTION

Therefore, the problem to be solved is to obtain a sun protecting cosmetic composition which comprises the UV-filter Bisoctrizole with a significantly reduced stickiness without reducing the sun protection factor, i.e. without reducing the amount of Bisoctrizole, of said composition.

Surprisingly, it has been found that the cosmetic composition according to claim 1 allows to solve this problem.

The composition comprising the specific mixture of branched and linear saturated C15-C19 alkanes and specific ether/ester emollients has a significant reduction of stickiness or sand adhesion, respectively without a reduction of Bisoctrizole. It has been particularly found that this solution represent a highly sustainable and advantageous approach for this problem as the preferred mixtures of C15-C19 alkanes can be obtained from biological origin. It is therefore, possible to offer cosmetic compositions having high sun protection factor (SPF) as well as significant reduction of stickiness.

Further aspects of the invention are subject of further independent claims. Particularly preferred embodiments are subject of dependent claims.

### DETAILED DESCRIPTION OF THE INVENTION

In a first aspect the present invention relates to a cosmetic composition comprising
a UV-filter of the formula (I)

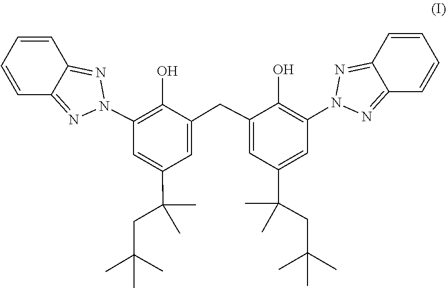 [SMILES: CC(C)(C)CC(C)(C)c1cc(Cc2cc(C(C)(C)CC(C)(C)C)cc(-n3nc4ccccc4n3)c2O)c(O)c(-n2nc3ccccc3n2)c1]

a C8-16 alkyl poly-glucoside
a mixture of branched and linear saturated C15-C19 alkanes
at least one emollient selected from the group consisting of dialkyl ether of the formula R¹OR¹, diester of a dicarboxylic acid of the formula R³OOCR²COOR³, aliphatic monoester of the formula R⁴COOR⁵ and ester of the formula R⁶COOR⁷ and wherein
   R¹ represents a C₅₋₁₄-alkyl group, particularly a C₆₋₁₀-alkyl group;
   R² represents a C₂₋₁₀-alkylene group, particularly a C₄₋₈-alkylene group, which optionally comprises at least one OH group;
   R³ represents a C₂₋₁₄-alkyl group, particularly a C₂₋₈-alkyl group;
   R⁴ represents a C₄₋₂₂-alkyl group, particularly a C₇₋₁₆-alkyl group;
   R⁵ represents a C₈₋₂₀-alkyl group, particularly a C₈₋₁₆-alkyl group, more particularly a C₁₀₋₁₆-alkyl group;
   R⁶ represents a C₆₋₁₀-aryl group, particularly a phenyl group;
   R⁷ represents a C₈₋₂₀-alkyl group, particularly a C₈₋₁₆-alkyl group, more particularly a C₁₂₋₁₅-alkyl group
wherein the amount of branched saturated C15-C19 alkane in said mixture of branched and linear saturated C15-C19 alkanes is more than 80% by weight, preferably more that 90% by weight, most preferred more than 92% by weight.

In the present document, a “C_{x-y}-alkyl” group is an alkyl group comprising x to y carbon atoms, i.e., for example, a C₁₋₃-alkyl group is an alkyl group comprising 1 to 3 carbon atoms. The alkyl group can be linear or branched. For example —CH(CH₃)—CH₂—CH₃ is considered as a C₄-alkyl group.

Analogously, a “C_{x-y}-alkylene” group is an alkylene group comprising x to y carbon atoms, i.e., for example, a C₁₋₃-alkylene group is an alkylene group comprising 1 to 3 carbon atoms. The alkylene group can be linear or branched. For example, —CH₂—CH₂—CH₂— and —CH(CH₃)—CH₂— and —C(CH₂—CH₃)— and —C(CH₃)₂— are all considered as a C₃-alkylene group.

In case identical labels for symbols or groups are present in several formulae, in the present document, the definition of said group or symbol made in the context of one specific formula applies also to other formulae which comprises the same said label.

The term “UV filter” in the present document stands for a substance that absorbs ultraviolet light (=UV light), i.e. electromagnetic radiation of the wavelength between 280 and 400 nm. UV(A) filters are UV filters that absorb UV(A) light, i.e. electromagnetic radiation of the wavelength between 315 and 400 nm. UV(B) filters are UV filters that absorb UV(B) light, i.e. electromagnetic radiation of the wavelength between 280 and 315 nm.

A liquid organic UV filter is liquid at ambient temperature (i.e. 25° C.).

A solid organic UV filter is solid at ambient temperature (i.e. 25° C.).

A “mixture of branched and linear saturated C15-C19 alkanes” in the present document means that said mixture comprises different alkanes each of them only having 15, 16, 17, 18 or 19 carbon atoms but does not comprise any alkanes having less carbons. Therefore, such a mixture does not contain for example dodecane or isododecane. Said mixture comprises both branched and linear C15-C19 alkanes.

### UV Filter of Formula (I)

The cosmetic composition comprises the UV-filter of the formula (I)

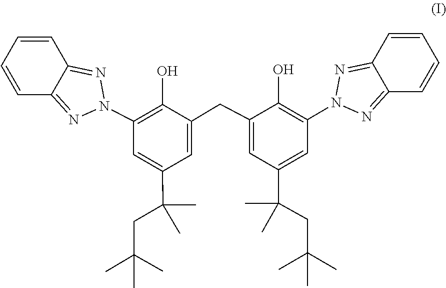 [SMILES: CC(C)(C)CC(C)(C)c1cc(Cc2cc(C(C)(C)CC(C)(C)C)cc(-n3nc4ccccc4n3)c2O)c(O)c(-n2nc3ccccc3n2)c1]

The UV-filter of the formula (I) (CAS: [103597-45-1]) is a solid with a melting point of 197-199° C. It is also known as Bisoctrizole or as methylene bis-benzotriazolyl tetramethylbutylphenol (INCI).

Bisoctrizole is a broad-spectrum ultraviolet radiation filter, absorbing UV(B) as well as UV(A) rays and has an excellent photostability. It has an absorption maximum at 308 nm and 349 nm. However, next to absorption of UV light, it also reflects and scatters UV light. Therefore, Bisoctrizole is a hybrid UV absorber, an organic UV filter produced in microfine organic particles (<200 nm). Where other organic UV filters need to be dissolved in either the oil or water phase, bisoctrizole dissolves poorly in both and is applied as invisible particle.

Bisoctrizole is mainly available as micronized powder in a 50% aqueous suspension which is stabilized by the surfactant decyl glucoside for example under the trademark PARSOL® Max from DSM Nutritional Products Ltd. or Tinosorb® M from BASF or as Eversorb M from Everlight Chemical or as Milestab 360. Most preferred is the product PARSOL® Max from DSM Nutritional Products Ltd.

Bisoctrizole has an extreme low solubility in most solvents or cosmetic oils and is almost insoluble, respectively very insoluble, particularly in water and alkanes.

Said composition has preferably a Sun Protection Factor (SPF) of 10 or higher, preferably of 20 or higher, more preferred of 30 or higher, even more preferred 50 or higher.

### Alkyl Poly-Glucoside

The cosmetic composition comprises a C8-C16 alkyl poly-glucoside.

Alkyl poly-glycosides (APGs) are a class of non-ionic surfactants widely used in a variety of household, cosmetic and industrial applications. APG's are derived from glucose of varying polymerization levels and fatty alcohols and have the generic formula CₙH₂₊ₙ O (C₆H₁₀O₅)ₓH, in which n is an integer selected in the range of 2 to 22 and x refers to the mean polymerization level of the glucoside moiety (mono-, di-, tri-, oligo-, and poly-glucoside). The raw materials for industrial manufacture thereof are typically corn derived glucose and plant derived fatty alcohols. The final products are typically complex mixtures of compounds with glucose moieties comprising the hydrophilic end and alkyl groups of variable length comprising the hydrophobic end.

In this application alkyl monoglucosides are also regarded as alkyl poly-glucosides.

The C₈₋₁₀ alkyl poly-glucosides generally exhibit a mean polymerisation level of the glucoside moiety ranging from 1 to 1.7, preferably from 1.1 to 1.6, most preferably from 1.1 to 1.4 such as in particular in the range of 1.1 to 1.3.

Further advantageous mean polymerisation level of the glucoside moiety range from 1.2 to 1.6 such as from 1.4 to 1.6. Additional advantageous mean polymerisation level of the glucoside moiety range from 1.2 to 1.7, respectively from 1.4 to 1.6.

Particularly advantageous C₈₋₁₀ alkyl poly-glucoside consist essentially of caprylyl (C₈) and capryl (C₁₀) poly-glucosides. Preferably such caprylyl (C₈) and capryl (C₁₀) poly-glucosides furthermore exhibit a ratio (%/%, wherein all % are area-% determined by HPLC-MS) of caprylyl (C₈) mono-glucoside to capryl (C₁₀) mono-glucoside in the range of 3:1 to 1:3, preferably in the range of about 2:1 to 1:2, most preferably in the range of 1.5:1 to 1:1.5. It is understood, that such C₈₋₁₀ alkyl poly-glucosides are preferably free of any (i.e. contain no) higher (i.e. C₁₄₋₁₆) alkyl polyglucosides

Thus, in an advantageous embodiment, the present invention also relates to the cosmetic composition according to present invention wherein the C₈₋₁₀alkyl poly-glucoside contains no more than 2% of C₁₂ alkyl mono-glucoside. It is further preferred that such C₈₋₁₀alkyl poly-glucosides do not contain any C₁₄₋₁₆ alkyl polyglucosides.

Furthermore, the C₈₋₁₀ alkyl poly-glucoside according to the invention consisting essentially of caprylyl (C₈) and capryl (C₁₀) poly-glucosides contains advantageously at least 60%, preferably at least 65%, most preferably at least 70% of the respective mono-glucosides as e.g. determined by HPLC-MS.

It is furthermore preferred that the C₈₋₁₀ alkyl poly-glucoside according to the present invention are substantially (i.e. essentially) free of any C₉ alkyl poly-glucosides, i.e. contain essentially no C₉ alkyl poly-glucosides. This means that the amount of any C₉ alkyl poly-glucosides in the C₈₋₁₀ alkyl poly-glucoside is less than 0.1 wt.-%, preferably less than 0.05 wt.-%, most preferably less than 0.01% such as in particular less than 0.005 wt.-%, based on the total weight of the C₈₋₁₀ alkyl poly-glucoside.

A particularly advantageous C₈₋₁₀ alkyl poly-glucoside according to the present invention is made from glucose derived from corn and C₈ and C₁₀ fatty alcohols derived from coconut and palm kernel oils, which is e.g. sold as an aqueous dispersion under the tradename Green APG 0810 by Shanghai Fine Chemical.

In one embodiment, the composition comprises a mixture of different alkyl poly-glucosides.

Particularly, preferred are products as offered as decyl glucoside (CAS: [68515-73-1]).

### Branched and Linear Saturated C15-C19 Alkanes

The cosmetic composition comprises a mixture of branched and linear saturated C15-C19 alkanes.

Particular suitable mixtures of C15-C19 alkanes are particularly the ones disclosed in WO 2016/185046, WO 2017/046177, WO 2018/109353 A1 and WO 2018/109354 A1 and WO 2018/172228 A1.

Preferably, the mixture of branched and linear saturated C15-C19 alkanes has a content of carbon of biological origin being greater or equal to 90% with respect of the total weight of the mixture of branched and linear saturated C15-C19 alkanes. The biological origin of chemicals is very advantageous as such material has a high degree of sustainability. High sustainable products or compositions are highly demanded in the market.

The determination of the content of biomaterial or content of biocarbon is given pursuant to standards ASTM D 6866-12, method B (ASTM D 6866-06) and ASTM D 7026 (ASTM D 7 026-04). Standard ASTM D 6866 concerns “Determi-ning the Biobased Content of Natural Range Materials Using Radiocarbon and Isotope Ratio Mass Spectrometry Analysis”, while standard ASTM D 7 026 concerns “Sampling and Reporting of Results for Determination of Biobased Content of Materials via Carbon Isotope Analysis”. The second standard mentions the first in its first paragraph. The first standard describes a test of measurement of the ratio ¹⁴C/¹²C of a sample and compares it with the ratio ¹⁴C/¹²C of a sample renewable reference of origin 100%, to give a relative percentage of C of origin renewable in the sample. The standard is based on the same concepts than the dating with ¹⁴C.

It is further preferred that the composition has no or a very small amount (less than 100 ppm, particularly less than 30 ppm) of aromatic hydrocarbons with respect to the total weight of the mixture of branched and linear saturated C15-C19 alkanes.

The mixture of branched and linear saturated C15-C19 alkanes is particularly produced by catalytic hydrogenation of hydrocarbon biomass feedstock, such as described in detail in WO 2016/185046, particular the one disclosed as example 3 of WO 2016/185046.

It is preferred that the amount of linear saturated C15-C19 alkanes in said mixture of branched and linear saturated C15-C19 alkanes is less than 10% by weight, preferably less than 8% by weight, most preferred more than 5% by weight.

It is further preferred that the amount of C15 is less than 3%, particularly less than 1%, preferably less than 0.05%, by weight in respect to the weight of said mixture of branched and linear saturated C15-C19 alkanes.

It is preferred that the mixture of branched and linear saturated C15-C19 alkanes is a mixture of branched and linear saturated C16-C19 alkanes.

It is further preferred that the amount of branched saturated C16-C18 alkane is more than 90% by weight, preferably more than 95% by weight in respect to the weight of said mixture of branched and linear saturated C15-C19 alkanes.

It is further preferred that the amount of C15 alkanes is less than 5%, particularly less than 2%, by weight in respect to the weight of said mixture of branched and linear saturated C15-C19 alkanes.

It is further preferred that the amount of branched saturated C17-C18 alkane is more than 85% by weight, preferably more than 92% by weight in respect to the weight of said mixture of branched and linear saturated C15-C19 alkanes.

It is further preferred that the amount of C17 alkanes is more between 15 and 20% by weight in respect to the weight of said mixture of branched and linear saturated C15-C19 alkanes.

It is further preferred that amount of branched saturated C18 alkane is more than 50% by weight, preferably more than 60% by weight, even more preferably more than 70% by weight, relative to the weight of said mixture of branched and linear saturated C15-C19 alkanes.

It is further preferred that the amount of C18 alkanes is particularly between 70 and 75% by weight in respect to the weight of said mixture of branched and linear saturated C15-C19 alkanes.

In other words, the mixture of branched and linear saturated C15-C19 alkanes consist preferably mainly of C18 alkane(s), most preferably mainly of branched C18 alkane(s).

As the cosmetic composition comprises a mixture of branched and linear saturated C15-C19 alkanes, said composition does not comprise any lower alkanes, i.e. it does particularly not comprise any C12 alkanes and particularly does not comprise any C12 or C13 or C14 alkanes.

It is further preferred that the mixture of C15-C19 alkanes has at 20° C., a viscosity of 3-15 mPa-s, particularly between 6 and 12 mPa-s.

It is further preferred that the mixture of C15-C19 alkanes has at 20° C. a refractive index of between 1.40 and 1.48, particularly of between 1.42 and 1.45, most preferably between 1.43 and 1.44.

It is further preferred that the mixture of C15-C19 alkanes is the mixtures of C15-C19 alkanes as commercialized as EMOGREEN™ L19 by SEPPIC.

### Emollient

The cosmetic composition comprises at least one emollient selected from the group consisting of dialkyl ether of the formula R¹OR¹, diester of a dicarboxylic acid of the formula R³COOR²COOR³, aliphatic monoester of the formula R⁴COOR⁵ and ester of the formula R⁶COOR⁷.
R¹ represents a C₅₋₁₄-alkyl group, particularly a C₆₋₁₀-alkyl group.
R² represents a C₂₋₁₀-alkylene group, particularly a C₄₋₈-alkylene group, which optionally comprises at least one OH group.
R³ represents a C₂₋₁₄-alkyl group, particularly a C₂₋₈-alkyl group.
R⁴ represents a C₄₋₂₂-alkyl group, particularly a C₇₋₁₆-alkyl group.
R⁵ represents a C₈₋₂₀-alkyl group, particularly a C₈₋₁₆-alkyl group, more particularly a C₁₀₋₁₆-alkyl group.
R⁶ represents a C₆₋₁₀-aryl group, particularly a phenyl group.
R⁷ represents a C₈₋₂₀-alkyl group, particularly a C₈₋₁₆-alkyl group, more particularly a C₁₂₋₁₅-alkyl group.

In a first embodiment, the emollient is a dialkyl ether of the formula R¹OR¹.

Preferably said dialkyl ether is selected from the group consisting of dihexylether, dioctylether, diethylhexylether, dioctylether and didecylether.

Preferably, the dialkyl ether of the formula R¹OR¹ is dioctylether (=dicaprylyl ether (INCI)).

In a second embodiment, the emollient is a diester of a dicarboxylic acid of the formula R³COOR²COOR³.

It is important to realize that this diester is a diester which is obtainable from an esterification of an dicarboxylic acid (═HOOC—R²⁻COOH) and an mono alcohol (R³—OH) and is not a diester obtainable from an esterification of a mono carboxylic acid and a diol.

Said dicarboxylic acid may comprise at least one OH group. Preferred example for dicarboxylic acids comprising hydroxyl group(s) are tartaric acid, pentaric acid and 3-hydroxyglutaric acid, preferably tartaric acid.

Particularly suitable dicarboxylic acids are selected from the group consisting of succinic acid, 2,2-dimethyl malonic acid, adipic acid, pimelic acid, sebacic acid, suberic acid, dodecanic acid, particularly from the group consisting of adipic acid, pimelic acid, sebacic acid and suberic acid. Most preferred said dicarboxylic acid is adipic acid or sebacic acid.

Said alcohol (R³—OH) is preferably selected from the group consisting of ethanol, propanol, iso-propanol, butanol, hexanol, octanol, 2-ethyhexanol, nonanol, iso-nonanol, decanol, iso-decanol, dodecanol and iso-dodecanol, preferably selected from the group consisting of ethanol, iso-propanol, butanol and 2-ethyhexanol.

Most preferred are ethanol, propanol, iso-propanol and butanol.

Diester of the formula R³OOCR²COOR³ are preferably diesters selected from the group consisting of diisopropyl sebacate, diethylhexyl adipate, dibutyl adipate, di-C12-13 alkyl tartrate, diethyl adipate and diisopropyl adipate.

Particularly preferred is diisopropyl sebacate.

In a third embodiment, the emollient is an aliphatic monoester of the formula R⁴COOR⁵.

Said ester is obtainable from an esterification of a carboxylic acid (═R⁴—COOH) and a mono alcohol (R⁵—OH).

Particularly suitable carboxylic acids are selected from the group consisting of pivalic acid, capronic acid, caprylic acid, 2-ethylhexanoic acid, capric acid, 3,5,5-trimethylhexanoic acid, isononanoic acid, lauric acid, myristic acid, palmitic acid, stearic acid and arachidonic acid, preferably from the group consisting of 2-ethylhexanoic acid, 3,5,5-trimethylhexanoic acid, isononanoic acid, lauric acid, myristic acid, palmitic acid and stearic acid.

Said alcohol (R⁵—OH) is preferably selected from the group consisting of octanol, 2-ethyhexanol, nonanol, iso-nonanol, decanol, iso-decanol, dodecanol iso-dodecanol, tridecanol iso-tridecanol and cetearyl alcohol. preferably selected from the group consisting of 2-ethyhexanol, iso-decanol, iso-tridecanol and cetearyl alcohol.

In one embodiment of preferred ester of the formula R⁴COOR⁵ the residue R⁴ represents a C₇₋₁₆-alkyl group and R⁵ represents a C₈₋₁₆-alkyl group, particularly R⁴=C₈-alkyl group and R⁵=C₁₀-alkyl group or C₁₃-alkyl group.

In another embodiment of preferred ester of the formula R⁴COOR⁵ the residue R⁴ represents a C₇₋₁₄-alkyl group and R⁵ represents a C₁₀₋₁₆-alkyl group, particularly R⁴=C₈-alkyl group and R⁵=C₁₀-alkyl group or C₁₃-alkyl group.

Esters of the formula R⁴COOR⁵ are preferably selected from the group consisting of ethylhexyl cocoate, ethylhexyl palmitate, isotridecyl myristate, isotridecyl isononanoate, isodecyl ethylhexanoate, isodecyl isononanoate, isodecyl octanoate, isodecyl neopentanoate and cetearyl isononanoate.

In a further embodiment, the emollient is an ester of the formula R⁶COOR⁷.

Preferably said ester is a C₈₋₂₀-alkyl ester, particularly a C₈₋₁₆-alkyl ester, more particularly a C₁₂₋₁₅-alkyl ester of benzoic acid. The ester of the formula R⁶COOR⁷ is most preferably C₁₂₋₁₅-alkyl benzoate.

The cosmetic composition may comprise two or more of the above emollients.

The composition comprises preferably at least two emollients, particularly comprises at least dicaprylyl ether and diisopropyl sebacate as emollients.

### Further Ingredients

The cosmetic composition typically comprises other ingredients which are suitable for the use in cosmetic compositions.

The cosmetic composition comprises preferably water.

The cosmetic compositions may be in the form of a suspension or dispersion in solvents or fatty substances, or alternatively in the form of an emulsion or micro emulsion (in particular of oil-in-water (O/W-) or water-in-oil (W/O-)type, silicone-in-water (Si/W-) or water-in-silicone (W/Si-)type, PIT-emulsion, multiple emulsion (e.g. oil-in-water-in oil (O/W/O-) or water-in-oil-in-water (W/O/W-)type), pickering emulsion, hydrogel, alcoholic gel, lipogel, one- or multiphase solution or vesicular dispersion or other usual forms, which can also be applied by pens, as masks or as sprays.

Preferred cosmetic compositions in all embodiments of the present invention comprise water and are in the form of an emulsion.

The emulsion particularly contain an oily phase and an aqueous phase such as in particular O/W, W/O, Si/W, W/Si, O/W/O, W/O/W multiple or a pickering emulsions.

The total amount of the oily phase present in such emulsions is preferably at least 10 wt.-%, such as in the range from 10 to 60 wt.-%, preferably in the range from 15 to 50 wt.-%, most preferably in the range from 15 to 40 wt.-%, based on the total weight of the cosmetic composition.

The amount of the aqueous phase present in such emulsions is preferably at least 20 wt. %, such as in the range from 40 to 90 wt.-%, preferably in the range from 50 to 85 wt.-%, most preferably in the range from 60 to 85 wt.-%, based on the total weight of the cosmetic composition.

More preferably, the cosmetic compositions are in the form of an oil-in-water (O/W) emulsion comprising an oily phase dispersed in an aqueous phase in the presence of an O/W-respectively Si/W-emulsifier. The preparation of such O/W emulsions is well known to a person skilled in the art.

The compositions in form of O/W emulsions can be provided, for example, in all the formulation forms for O/W emulsions, for example in the form of serum, milk or cream, and they are prepared according to the usual methods. The compositions are preferably intended for topical application and can in particular constitute a dermatological or cosmetic composition, for example intended for protecting human skin against the adverse effects of UV radiation (antiwrinkle, anti-ageing, moisturizing, sun protection and the like).

The cosmetic composition comprises preferably a further UV filter. The further UV filter may be solid or liquid. It is preferred that the further UV filter is a solid UV filter.

Suitable liquid organic UV-filter absorb light in the UV(B) and/or UV(A) range and are liquid at ambient temperature (i.e. 25° C.). Such liquid UV-filter are well known to a person in the art and encompass in particular cinnamates such as e.g. octyl methoxycinnamate (PARSOL® MCX) and isoamyl methoxycinnamate (Neo Heliopan® E 1000), salicylates such as e.g. homosalate (3,3,5 trimethyl-cyclohexyl 2-hydroxybenzoate, PARSOL® HMS) and ethylhexyl salicylate (also known as ethylhexyl salicylate, 2-ethylhexyl 2-hydroxybenzoate, PARSOL® EHS), acrylates such as e.g. octocrylene (2-ethylhexyl-2-cyano-3,3-diphenylacrylate, PARSOL® 340) and ethyl 2-cyano-3,3 diphenylacrylate, esters of benzalmalonic acid such as in particular dialkyl benzalmalonates such as e.g. di (2-ethylhexyl) 4-methoxybenzalmalonate and polysilicone 15 (PARSOL® SLX), dialkylester of naphthalates such as e.g. diethylhexyl 2,6-naphthalate (Corapan® TQ), syringylidene malonates such as e.g. diethylhexyl syringylidene malonate (Oxynex® ST liquid) as well as benzotriazolyl dodecyl p-cresol (Tinoguard® TL) as well as benzophenone-3 and drometrizole trisiloxane.

Particular advantageous liquid organic UV-filter are octyl methoxycinnamate, homosalate, ethylhexyl salicylate, octocrylene, diethylhexyl-2,6-naphthalate, diethylhexyl syringylidene malonate, benzotriazolyl dodecyl p-cresol, benzo-phenone-3, drometrizole trisiloxane as well as mixtures thereof.

In a preferred embodiment, the liquid UV filter is a liquid UV(B) filter which is selected from the group consisting of ethylhexyl methoxycinnamate, octocrylene, homosalate, ethylhexyl salicylate, benzophenone-3 and drometrizole trisiloxane.

Suitable solid organic UV-filter absorb light in the UV(B) and/or UV(A) range and are solid at ambient temperature (i.e. 25° C.). Particularly suited solid UV-filters are of the group consisting of bis-ethylhexyloxyphenol methoxyphenyl triazine, butyl methoxydibenzoyl methane, diethylamino hydroxybenzoyl hexyl benzoate, ethylhexyl triazone, diethylhexyl butamido triazone, 4-methylbenzylidene camphor and 1,4-di(benzoxazol-2′-yl)benzene.

A preferred solid organic UV(A) filter is a UV(A) filter which is selected from the group consisting of bis-ethylhexyloxyphenol methoxyphenyl triazine, butyl methoxydibenzoyl methane, diethylamino hydroxybenzoyl hexyl benzoate and tris-biphenyl triazine.

A preferred solid organic UV(B) filter is a UV(B) filter which is selected from the group consisting of ethylhexyl triazone (=Uvinul® T150), diethylhexyl butamido triazone (=Uvasorb® HEB), and 4-methylbenzylidene camphor (=PARSOL® 5000).

The total amount of organic UV filter (s) depends strongly on the targeted UV protection.

It is preferred that the amount of a solid organic UV filter, particular of solid organic UV(A) filter, is selected in the range of 0.1 to about 6 wt.-%, preferable in the range of 0.5 to 5 wt.-%, most preferably in the range of 1 to 4 wt.-%.

It is further preferred that amount of a solid organic UV filter, particular of solid organic UV(B) filter, is selected in the range of 0.1 to about 6 wt.-%, preferable in the range of 0.5 to 5 wt.-%, most preferably in the range of 1 to 4 wt.-%.

It is even further preferred that amount of a liquid organic UV filter, particular of liquid organic UV(B) filter, is selected in the range of 0.1 to about 10 wt.-%, preferable in the range of 0.5 to 12 wt.-%, most preferably in the range of 1 to 10 wt.-%.

It is preferred that the weight ratio of UV-filter of the formula (I) to the further UV filter is between 1:10 and 5:1, preferably between 1:5 and 3:1, more preferably between 1:3 and 1:1.

In an embodiment, the cosmetic composition further comprises preferably an ester of a fatty acid and dextrin.

Dextrin is an oligomer polymers of D-glucose. Its structure can be represented simplified by the following structure

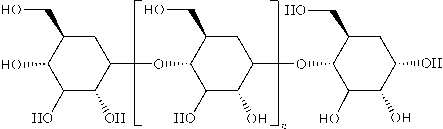

Dextrins have different average degrees of glycopolymerization which leads to different molecular weights.

In the present invention, the dextrin of said ester of a fatty acid and dextrin has preferably an average degree of glycopolymerization of between 3 and 20, particularly between 8 and 16.

It is preferred that the fatty acid of said ester of a fatty acid and dextrin is a C14-C18 fatty acid, particularly a linear C14-C18 fatty acid, most preferably palmitic acid.

As particular suitable ester of a fatty acid and dextrin is a dextrin palmitate as commercialized as Rheopearl® KL2 by Chiba Flour Milling.

Dextrin has several hydroxyl groups which can be esterified.

It is preferred that said ester of a fatty acid and dextrin has an average number of esterified hydroxyl groups of more than 2.5, preferably between 2.7 and 3.5, more preferably between 28 and 3.4, most preferably between 2.8 and 3.2, per glucose unit.

In one embodiment said ester of a fatty acid and dextrin has an average number of esterified hydroxyl groups of more than 3, preferably between 3.05 and 3.5, more preferably between 3.1 and 3.4, most preferably between 3.1 and 3.2, per glucose unit.

In other words, preferably essentially all of the hydroxyl groups of the dextrin are esterified.

It is further preferred that said ester of a fatty acid and dextrin has an molecular weight Mn of between 8,000 and 16,000 Da, preferably between 9,000 and 13,000 Da, more preferably between 10,000 and 11,500 Da.

The molecular weight Mn is determined in Dalton (Da) particularly by SEC/GPC using polystyrene as standard.

It is preferred that the ratio of the weight of said ester of a fatty acid and dextrin to the weight of said mixture of branched and linear saturated C15-C19 alkanes is preferably less than 100% by weight, preferably in the range of 50-80% by weight, most preferred in the range of 60-70% by weight.

Both fatty acid and dextrin have biological origin. The biological origin of chemicals is very advantageous as such material or products thereof have a high degree of sustainability. High sustainable products or compositions are highly demanded in the market.

The cosmetic composition further preferably comprises at least one emulsifier, preferably an anionic emulsifier. Preferably the anionic emulsifier is an anionic emulsifier selected from the group consisting of potassium cetyl phosphate, disodium cetearyl sulfosuccinate, sodium stearoyl glutamate, sodium stearoyl lactylate, glyceryl stearate citrate and sodium cocoyl isethionate.

In one advantageous embodiment, the cosmetic compositions in addition contain a phosphate ester emulsifier. Among the preferred phosphate ester emulsifier are C8-10 Alkyl Ethyl Phosphate, C9-15 Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C12-15 Pareth-2 Phosphate, C12-Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. A particular phosphate ester emulsifier is potassium cetyl phosphate e.g. commercially available as Amphisol® K at DSM Nutritional Products Ltd Kaiseraugst.

The cosmetic composition can also comprise nonionic emulsifiers.

Examples of nonionic emulsifier include condensation products of aliphatic (C8-C18) primary or secondary linear or branched chain alcohols with alkylene oxides, usually ethylene oxide and generally having from 6 to 30 ethylene oxide groups. Other representative nonionic emulsifiers include mono- or di-alkyl alkanolamides such as e.g. coco mono- or di-ethanolamide and coco mono-iso-propanolamide. Further nonionic emulsifiers which can be included are the alkyl polyglycosides (APGs). Typically, the APG is one which comprises an alkyl group connected (optionally via a bridging group) to a block of one or more glycosyl groups such as e.g. Oramix™ NS 10 ex Seppic; PLANTACARE® 818UP, PLANTACARE® 1200 and PLANTACARE® 2000 ex BASF.

If the cosmetic composition is an O/W emulsion, then it preferably contains at least one O/W- or Si/W-emulsifier selected from the list of PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, glycerylstearatcitrate, glycerylstearate (self-emulsifying), stearic acid, salts of stearic acid, polyglyceryl-3-methylglycosedistearate. Further suitable emulsifiers are sorbitan oleate, sorbitan sesquioleate, sorbitan isostearate, sorbitan trioleate, Lauryl Glucoside, Decyl Glucoside, Sodium Stearoyl Glutamate, Sucrose Polystearate and Hydrated Polyisobuten.

Furthermore, one or more synthetic polymers may be used as an emulsifier. For example, PVP eicosene copolymer, acrylates/C10-30 alkyl acrylate crosspolymer, acrylates/steareth-20 methacrylate copolymer, PEG-22/dodecyl glycol copolymer, PEG-45/dodecyl glycol copolymer, and mixtures thereof.

Another particular suitable class of O/W emulsifiers are non-ionic self-emulsifying system derived from olive oil e.g. known as (INCI Name) cetearyl olivate and sorbitan olivate (Chemical Composition: sorbitan ester and cetearyl ester of olive oil fatty acids) sold under the tradename OLIVEM 1000.

Further suitable are commercially available polymeric emulsifiers such as hydrophobically modified polyacrylic acid such as Acrylates/C10-30 Alkyl Acrylate Crosspolymers which are commercially available under the tradename Pemulen® TR-1 and TR-2 by Noveon.

Another class of particularly suitable emulsifiers are polyglycerol esters or diesters of fatty acids also called polyglyceryl ester/diester (i.e. a polymer in which fatty acid(s) is/are bound by esterification with polyglycerine), such as e.g. commercially available at Evonik as Isolan GPS [INCI Name Polyglyceryl-4 Diisostearate/Polyhydroxystearate/Sebacate (i.e. diester of a mixture of isostearic, polyhydroxystearic and sebacic acids with Polyglycerin-4)] or Dehymuls PGPH available at Cognis (INCI Polyglyceryl-2 Dipolyhydroxystearate).

Also suitable are polyalkylenglycolether such as Brij 72 (Polyoxyethylen(2)stearylether) or Brij 721 (Polyoxyethylene (21) Stearyl Ether e.g. available at Croda.

The at least one O/W respectively Si/W emulsifier is preferably used in an amount of 0.5 to 10 wt. % such as in particular in the range of 0.5 to 5 wt.-% such as most in particular in the range of 0.5 to 4 wt.-% based on the total weight of the composition.

Suitable W/O- or W/Si-emulsifiers are polyglyceryl-2-dipolyhydroxystearat, PEG-30 dipolyhydroxystearat, cetyl dimethicone copolyol, polyglyceryl-3 diisostearate polyglycerol esters of oleic/isostearic acid, polyglyceryl-6 hexaricinolate, polyglyceryl-4-oleate, polygylceryl-4 oleate/PEG-8 propylene glycol cocoate, magnesium stearate, sodium stearate, potassium laurate, potassium ricinoleate, sodium cocoate, sodium tallowate, potassium castorate, sodium oleate, and mixtures thereof. Further suitable W/Si-emulsifiers are Lauryl Polyglyceryl-3 Polydimethylsiloxyethyl Dimethicone and/or PEG-9 Polydimethylsiloxyethyl Dimethicone and/or Cetyl PEG/PPG-10/1 Dimethicone and/or PEG-12 Dimethicone Crosspolymer and/or PEG/PPG-18/18 Dimethicone. The at least one W/O emulsifier is preferably used in an amount of about 0.001 to 10 wt.-%, more preferably in an amount of 0.2 to 7 wt.-% with respect to the total weigh of the composition.

The cosmetic compositions according preferably furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 6 wt.-%, such as most in particular in the range of 1 to 5 wt.-%, based on the total weight of the composition. Particular suitable co-surfactants are selected from the list of alkyl alcohols such as cetyl alcohol (Lorol C16, Lanette 16), cetearyl alcohol (Lanette O), stearyl alcohol (Lanette 18), behenyl alcohol (Lanette 22), glyceryl stearate, glyceryl myristate (Estol 3650), hydrogenated coco-glycerides (Lipocire Na10) as well as mixtures thereof.

The amount of emulsifier is preferably in the range between 0.1-6.0% by weight, more preferably between 0.25-5.0% by weight, particularly between 0.5-4.0% by weight, based on the total weight of the cosmetic composition.

The composition is preferably sulfate-free.

Hence, the cosmetic composition is preferably particularly free of sulfates of the group consisting of alkyl sulfates, alkyl ether sulfates, alkyl amido ether sulfates, alkylaryl polyether sulfates and monoglycerides sulfate as well as mixtures thereof.

The term “free” as used in the present document, for example in “sulfate-free”, is used to mean that the respective substance is only present at amounts of less than 0.5% by weight, particularly less than 0.1% by weight, more particularly below 0.05% by weight, relative to the weight of the composition. Preferably, “free” means that the respective substance is completely absent in the composition.

The term “sulfate-free” is used in the present document to mean that the composition is free of any anionic tenside having a terminal anionic group of the formula

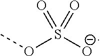

The cosmetic composition is preferably free of cationic emulsifiers. Typical example for such cationic emulsifiers are isostearamidopropyl dimethylamine, stearalkonium chloride, stearamidoethyl diethylamine, behentrimonium methosulfate, behenoyl PG-trimonium chloride, cetrimonium bromide, behenamidopropyl dimethylamine behenate, brassicamidopropyl dimethylamine, stearamidopropyl dimethylamine stearate, cocamidopropyl PG-dimonium chloride, distearoylethyl hydroxyethylmonium methosulfate, dicocoylethyl hydroxyethylmonium methosulfate, distearoylethyl dimonium chloride, shea butteramidopropyltrimonium chloride, behenamidopropyl dimethylamine, brassicyl isoleucinate esylate, acrylamidopropyltrimonium chloride/acrylates copolymer, linoleamidopropyl ethyldimonium ethosulfate, dimethyl lauramine isostearate, isostearamidopropyl laurylacetodimonium chloride, particularly behentrimonium chloride, distearyldi-monium chloride, cetrimonium chloride, steartrimonium chloride, and palmitamido-propyltrimonium chloride.

The cosmetic composition further may comprise cosmetic carriers, excipients and diluents as well as additives and active ingredients commonly used in the skin care industry which are suitable for use in the cosmetic compositions are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

Such possible ingredients of the cosmetic composition are particularly enhance the performance and/or consumer acceptability such as preservatives, antioxidants, fatty substances/oils, thickeners, softeners, light-screening agents, moisturizers, fragrances, co-surfactants, fillers, sequestering agents, cationic-, nonionic- or amphoteric polymers or mixtures thereof, acidifying or basifying agents, viscosity modifiers, and natural hair nutrients such as botanicals, fruit extracts, sugar derivatives and/or amino acids or any other ingredients usually formulated into cosmetic compositions. The necessary amounts of the adjuvants and additives can, based on the desired product, easily be chosen by a person skilled in the art in this field and will be illustrated in the examples, without being limited hereto.

Particularly suitable thickeners in all embodiments are xanthan gum, gellan gum and/or carboxymethylcellulose. Most preferably in all embodiments the thickener is xanthan gum or gellan gum.

Such thickener(s) are preferably used in an amount (total) selected in the range from 0.1 to 1 wt.-%, more preferably in an amount of 0.1 to 0.5 wt.-%, based on the total weight of the cosmetic composition.

The cosmetic compositions preferably have a pH in the range from 3 to 10, preferably a pH in the range from 4 to 8 and most preferably a pH in the range from 4 to 7.5. The pH can easily be adjusted as desired with suitable acids such as e.g. citric acid or bases such as NaOH according to standard methods in the art.

The cosmetic composition is preferably sulfate-free and/or free of para-bens, and/or silicone oils and/or silicone surfactants.

The cosmetic composition is preferably a topical composition.

The term “topical” as used herein is understood here to mean external application to keratinous substances, which are in particular the skin, scalp, eyelashes, eyebrows, nails, mucous membranes and hair, preferably the skin.

As the topical compositions are intended for topical application, it is well understood that they comprise a physiologically acceptable medium, i.e. a medium compatible with keratinous substances, such as the skin, mucous membranes, and keratinous fibers. In particular, the physiologically acceptable medium is a cosmetically acceptable carrier.

The term “cosmetically acceptable carrier” refers to all carriers and/or excipients and/or diluents conventionally used in cosmetic compositions such as in particular in sun care products.

Preferably the cosmetic composition is a skin care preparation, decorative preparation, or a functional preparation.

Examples of skin care preparations are, in particular, light protective preparations, anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples of functional preparations are cosmetic or pharmaceutical compositions containing active ingredients such as hormone preparations, vitamin preparations, vegetable extract preparations and/or anti-ageing preparations without being limited thereto.

The cosmetic composition is preferably a skin care composition.

In a most preferred embodiment, the cosmetic composition is a sun care composition. Sun care compositions are light-protective preparations (sun care products), such as sun protection milks, sun protection lotions, sun protection creams, sun protection oils, sun blocks or day care creams with a SPF (sun protection factor). Of particular interest are sun protection creams, sun protection lotions, sun protection milks and sun protection preparations.

The cosmetic compositions have improved sensory properties, particular improved afterfeel.

In one of the embodiments, the cosmetic composition is in the form of a gel.

It has been shown that the above cosmetic compositions have a reduced stickiness. It has been observed that the stickiness after a prolonged time, i.e. more than 3 minutes, particularly more than 15 minutes, after the application of the cosmetic composition has been significantly reduced. It has been shown that the above cosmetic composition have a reduced stickiness. It is has been observed that the skin to which the cosmetic composition is applied is significant less sticky, particularly less sticky to sand. This is particularly important for its use at a beach.

It has been shown that by adding said mixture of branched and linear saturated C15-C19 alkanes as mentioned above to cosmetic composition comprising the UV-Filter Bisoctrizole, and particularly the ether and ester based emollients as described above in great detail reducing its stickiness.

Hence, in a further aspect, the present invention relates to the use of a mixture of branched and linear saturated C15-C19 alkanes for reducing the stickiness of a composition comprising the UV-Filter Bisoctrizole and a C8-C16 alkyl poly-glucoside, wherein the amount of branched saturated C15-C19 alkane in said mixture of branched and linear saturated C15-C19 alkanes is more than 80% by weight, preferably more that 90% by weight, most preferred more than 92% by weight.

Preferably, in said use an at least one emollient selected from the group consisting of dialkyl ether of the formula R¹OR¹, diester of a dicarboxylic acid of the formula R³OOCR²COOR³, and aliphatic monoester of the formula R⁴COOR⁵ and an ester of the formula R⁶COOR⁷, more preferably selected from the group consisting of dialkyl ether of the formula R¹OR¹, diester of a dicarboxylic acid of the formula R³OOCR²COOR³, and aliphatic monoester of the formula R⁴COOR⁵, is involved.

The definitions and preferences of the ingredients have already been described above in great details.

Furthermore, in a further aspect, the present invention relates to the use of a mixture of branched and linear saturated C15-C19 alkanes for reducing the stickiness of the skin and/or reducing the sand adherence, particularly reducing the sand adherence, onto the skin to which a composition comprising the UV-Filter Bisoctrizole and a C8-C16 alkyl poly-glucoside has been applied; wherein the amount of branched saturated C15-C19 alkane in said mixture of branched and linear saturated C15-C19 alkanes is more than 80% by weight, preferably more that 90% by weight, most preferred more than 92% by weight.

Preferably, in said use an at least one emollient selected from the group consisting of dialkyl ether of the formula R¹OR¹, diester of a dicarboxylic acid of the formula R³OOCR²COOR³, and aliphatic monoester of the formula R⁴COOR⁵ and an ester of the formula R⁶COOR⁷, more preferably selected from the group consisting of dialkyl ether of the formula R¹OR¹, diester of a dicarboxylic acid of the formula R³OOCR²COOR³, and aliphatic monoester of the formula R⁴COOR⁵, is involved.

The definitions and preferences of the ingredients have already been described above in great details.

### EXAMPLES

The following examples are provided to further illustrate the compositions and effects of the present invention. These examples are illustrative only and are not intended to limit the scope of the invention in any way.

The cosmetic composition as outlined in table 1 or table 2 have been prepared according to standard methods in the art. Then the sand repellence has been tested according to the method as outlined below:
Apply 50 mg of the cosmetic composition on PMMA plate (2 μm roughness) and distribute homogenously (2 mg/cm²)
Let it dry for 15 minutes
Weigh the PMMA plate (m_{plate+film})
Put sand (Sigma, product no. 84878) in a petri dish
place the plate with the film side on the sand to contact film with sand
put a weight (500 g) and wait for 5 minutes
Take the plate out and the PMMA plate and turn around 180° without shaking
Weigh the PMMA plate (m_{plate+film+sand})
Calculate the amount of sand sticking (m_{sand}) on the plate by

m_{sand}=m_{plate+film+sand}−m_{plate+film}
Repeat test with 4 plates per sample
   Furthermore, the %-change of adherent sand in comparison with Ref.1 (Δ_{msand, Ref.1}), or with Ref.3 (Δ_{msand, Ref.3}), respectively, have been calculated.

All compositions are homogenous directly after preparation. However, after a certain time of standing at room temperature, some separations can be observed. Such separation can be assessed by eye. The longer the composition is homogenous the better the stability is. The stability of a composition being assessed as “++” is significantly higher than the one of a composition being assessed as “+”.

The viscosity has been measured with a Rheometer TA HR10. The geometry are 40 mm plates and the used shear rate was 10/s. All measurements have been done at 25° C. and the results are given in mPas.

The results are given in table 1 and table 2.

TABLE 1
Cosmetic composition and stickiness to sand.

| INCI | Ref. 1 | Ref. 2 | 1 | 2 | 3 |
|---|---|---|---|---|---|
| C12-15 Alkyl Benzoate [wt. %] | 15.0 | 15.0 | 12.0 | 12.0 | 12.0 |
| Cetearyl Alcohol [wt. %] | 2.0 | 2.0 | 2.0 | 2.0 | 2.0 |
| C15-19 Alkane [wt. %]¹ | | | 3.0 | 3.0 | 3.0 |
| Dextrin Palmitate 1 [wt. %]² | | 2.0 | | 2.0 | |
| Dextrin Palmitate 2 [wt. %]³ | | | | | 2.0 |
| Potassium Cetyl Phosphate [wt. %] | 2.0 | 2.0 | 2.0 | 2.0 | 2.0 |
| Propanediol [wt. %] | 2.0 | 2.0 | 2.0 | 2.0 | 2.0 |
| Xanthan Gum [wt. %] | 0.25 | 0.25 | 0.25 | 0.25 | 0.25 |
| Preservative (phenoxyethanol) [wt. %] | 1.0 | 1.0 | 1.0 | 1.0 | 1.0 |
| Water [wt. %] | 67.75 | 65.75 | 67.75 | 65.75 | 65.75 |
| PARSOL ® MAX [wt. %]⁴ | 10.0 | 10.0 | 10.0 | 10.0 | 10.0 |
| m_{sand}[g] | 100.9 | 146.2 | 79.0 | 102.7 | 113.0 |
| Δm_{sand, Ref. 1} [%] | | 45 | −22 | 2 | 12 |
| Viscosity [mPas] | 682 | 749 | 596 | 727 | 735 |
| Stability | + | ++ | + | ++ | ++ |

¹EMOGREEN ™ L19
²Rheopearl ® KL2, Chiba Flour Milling
³Dextrin Palmitate: Mₙ = 11′300-11′500 Da, determined by SEC/GPC.
⁴PARSOL ® MAX: Bisoctrizole stabilized by decyl glucoside.

TABLE 2
Cosmetic composition and stickiness to sand.

| INCI | Ref. 3 | Ref. 4 | 4 | 5 | 6 |
|---|---|---|---|---|---|
| C12-15 Alkyl Benzoate [wt. %] | 25.0 | 25.0 | 19.0 | 19.0 | 19.0 |
| Cetearyl Alcohol [wt. %] | 7.0 | 3.0 | 7.0 | 3.0 | 3.0 |
| C15-19 Alkane [wt. %]¹ | | | 6.0 | 6.0 | 6.0 |
| Dextrin Palmitate 1 [wt. %]² | | 4.0 | | 4.0 | |
| Dextrin Palmitate 2 [wt. %]³ | | | | | 4.0 |
| Potassium Cetyl Phosphate [wt. %] | 2.0 | 2.0 | 2.0 | 2.0 | 2.0 |
| Propanediol [wt. %] | 2.0 | 2.0 | 2.0 | 2.0 | 2.0 |
| Xanthan Gum [wt. %] | 0.25 | 0.25 | 0.25 | 0.25 | 0.25 |
| Preservative (phenoxyethanol) [wt. %] | 1.0 | 1.0 | 1.0 | 1.0 | 1.0 |
| Water [wt. %] | 52.75 | 52.75 | 52.75 | 52.75 | 52.75 |
| PARSOL ® MAX [wt. %]⁴ | 10.00 | 10.00 | 10.00 | 10.00 | 10.00 |
| m_{sand}[g] | 70.4 | 241.2 | 38.3 | 164.8 | 151.9 |
| Δm_{sand, Ref. 3} [%] | | 242 | −46 | 134 | 116 |
| Viscosity [mPas] | | 2928 | 2220 | 2323 | 2575 |
| Stability | + | ++ | + | ++ | ++ |

¹EMOGREEN ™ L19
²Rheopearl ® KL2, Chiba Flour Milling
³Dextrin Palmitate: Mₙ = 11′300-11′500 Da, determined by SEC/GPC.
⁴PARSOL ® MAX: Bisoctrizole stabilized by decyl glucoside.

As can be seen from tables 1 and 2, the cosmetic composition comprising Bisoctrizole show a very high stickiness to sand (Ref.1 and Ref.3). The experiments show that the use of 015-19 alkane mixture reduces dramatically the sand adherence (1 vs Ref.1 and 4 vs Ref.3). The use of an ester of fatty acid and dextrin increases the stickiness significantly (Ref.2 vs Ref.1 and Ref.4 vs Ref.3). However, when using an ester of fatty acid and dextrin, the stability of the composition is increased (Ref.2 vs Ref.1 and 2 and 3 vs 1) (Ref.4 vs Ref.3 and 5 and 6 vs 4)).

All compositions of table 1 or table 2, respectively, are very similar in viscosity, however, generally when an ester of fatty acid and dextrin is added, the viscosity increases (e.g. Ref.1 vs Ref.2 or 1 vs 2,3 or 4 vs 5 or 6), whereas by adding the 015-19 alkane mixture the viscosity is reduced (1 vs Ref.1). A combination of ester of fatty acid and dextrin and C15-19 alkane mixture allows keeping the viscosity more or less constant.

It can be also seen from the results of tables 1 and 2, that the sand adherence of a composition comprising a C15-19 alkane mixture and an ester of fatty acid and dextrin (i.e. 2,3 resp. 5, 6) is significantly lower than one would expect from the values of the respective individual compositions comprising a C15-19 alkane mixture or an ester of fatty acid and dextrin (i.e. Ref.2,1 resp. Ref.4,4): e.g.
2: 2% is significantly lower than 33% (=45% (Ref.2)−22% (1)) or
5: 134% is significantly lower than 196% (=242% (Ref.4) −46% (4)).

This finding combined with the finding of higher stability when using a C15-19 alkane mixture and an ester of fatty acid and dextrin is very advantageous for a cosmetic composition comprising Bisoctrizole (i.e. 2,3,5,6)

1. A cosmetic composition comprising
a UV-filter of the formula (I) 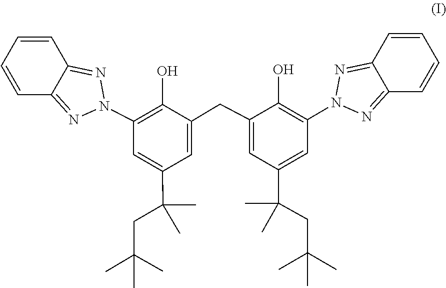 [SMILES: CC(C)(C)CC(C)(C)c1cc(Cc2cc(C(C)(C)CC(C)(C)C)cc(-n3nc4ccccc4n3)c2O)c(O)c(-n2nc3ccccc3n2)c1]
C8-C16 alkyl poly-glucoside
a mixture of branched and linear saturated C15-C19 alkanes
at least one emollient selected from the group consisting of dialkyl ether of the formula R¹OR¹, diester of a dicarboxylic acid of the formula R³OOCR²COOR³, aliphatic monoester of the formula R⁴COOR⁵ and ester of the formula R⁶COOR⁷ and wherein
R¹ represents a C₅₋₁₄-alkyl group, particularly a C₆₋₁₀-alkyl group;
R² represents a C₂₋₁₀-alkylene group, particularly a C₄₋₈-alkylene group, which optionally comprises at least one OH group;
R³ represents a C₂₋₁₄-alkyl group, particularly a C₂₋₈-alkyl group;
R⁴ represents a C₄₋₂₂-alkyl group, particularly a C₇₋₁₆-alkyl group;
R⁵ represents a C₈₋₂₀-alkyl group, particularly a C₈₋₁₆-alkyl group, more particularly a C₁₀₋₁₆-alkyl group;
R⁶ represents a C₆₋₁₀-aryl group, particularly a phenyl group;
R⁷ represents a C₈₋₂₀-alkyl group, particularly a C₈₋₁₆-alkyl group, more particularly a C₁₂₋₁₅-alkyl group;
wherein the amount of branched saturated C15-C19 alkane in said mixture of branched and linear saturated C15-C19 alkanes is more than 80% by weight, preferably more that 90% by weight, most preferred more than 92% by weight.

2. The composition according to claim 1, characterized in that the amount of linear saturated C15-C19 alkanes in said mixture of branched and linear saturated C15-C19 alkanes is less than 10% by weight, preferably less than 8% by weight, most preferred more than 5% by weight.

3. The composition according to claim 1, characterized in that amount of branched saturated C18 alkane is more than 50% by weight, preferably more than 60% by weight, even more preferably more than 70% by weight, relative to the weight of said mixture of branched and linear saturated C15-C19 alkanes.

4. The composition according to claim 1 characterized in that the emollient is dicaprylyl ether.

5. The composition according to claim 1 characterized in that the emollient is diisopropyl sebacate.

6. The composition according to claim 1 characterized in that the composition comprises at least two emollients, particularly comprises at least dicaprylyl ether and diisopropyl sebacate as emollients.

7. The composition according to claim 1 characterized in that the C8-C16 alkyl poly-glucoside is decyl glucoside (CAS: [68515-73-1]).

8. The composition according to claim 1 characterized in that the composition further comprises at least one further UV-filter, particularly so that the weight ratio of UV-filter of the formula (I) to the further UV filter, is between 1:15 and 5:1, preferably between 1:10 and 4:1, more preferably between 1:8 and 3:1.

9. The composition according to claim 1 characterized in that the composition further comprises an ester of a fatty acid and dextrin.

10. The composition according to claim 9 characterized in that the fatty acid of said ester of a fatty acid and dextrin is a C14-C18 fatty acid, particularly a linear C14-C18 fatty acid, most preferably palmitic acid.

11. The composition according to claim 1 characterized in that the composition comprises water and is in the form of an emulsion.

12. The composition according to claim 1 characterized in that the composition is in the form of a gel.

13. The composition according to claim 1 characterized in that the composition has a Sun Protection Factor (SPF) of 10 or higher, preferably of 20 or higher, more preferred of 30 or higher, even more preferred 50 or higher.

14. Use of a mixture of branched and linear saturated C15-C19 alkanes for reducing the stickiness of a composition comprising the UV-Filter Bisoctrizole and a C8-C16 alkyl poly-glucoside, wherein the amount of branched saturated C15-C19 alkane in said mixture of branched and linear saturated C15-C19 alkanes is more than 80% by weight, preferably more that 90% by weight, most preferred more than 92% by weight.

15. Use of a mixture of branched and linear saturated C15-C19 alkanes for reducing the stickiness of the skin and/or reducing the sand adherence, particularly reducing the sand adherence, onto the skin to which a composition comprising the UV-Filter Bisoctrizole and a C8-C16 alkyl poly-glucoside has been applied;
wherein the amount of branched saturated C15-C19 alkane in said mixture of branched and linear saturated C15-C19 alkanes is more than 80% by weight, preferably more that 90% by weight, most preferred more than 92% by weight.
